(19) United States
(12) Patent Application Publication
(10) Pub. No.: US 2024/0315778 A1
(43) Pub. Date: Sep. 26, 2024

(54) SURGICAL ASSISTANCE SYSTEM AND DISPLAY METHOD

(71) Applicant: B. Braun New Ventures GmbH, Freiburg im Breisgau (DE)

(72) Inventors: Amir Sarvestani, Freiburg (DE); Rudolf Zepf, Wurmlingen (DE); Matthias Kuehn, Freiburg (DE); Niels Lemke, Emmendingen (DE); Stefan Schroeer, Freiburg (DE)

(21) Appl. No.: 18/276,795

(22) PCT Filed: Feb. 4, 2022
(86) PCT No.: PCT/EP2022/052730

§ 371 Date: Aug. 10, 2023

(30) Foreign Application Priority Data
Feb. 12, 2021 (DE) 10 2021 103 411.6

(51) Int. Cl. A61B 34/20; A61B 1/00; A61B 5/06; A61B 34/30; A61B 90/00

(52) U.S. Cl. CPC A61B 34/20; A61B 1/00009; A61B 1/00045; A61B 5/067; A61B 2034/2065; A61B 2034/301; A61B 2090/367

(57) ABSTRACT

A surgical assistance system, method for displaying a recording, a storage medium, and a sterile space. The surgical assistance system includes a display device; an endoscope with a distal imaging recording head adapted to create an intracorporeal recording of the patient; a data-providing unit adapted to provide digital 3D recording data of the patient; and a control unit adapted to process the intracorporeal recording and the 3D recording data. The intracorporeal recordings are registered with the 3D recording data initially via corresponding anatomical landmarks and/or orientations. A tracking system continuously detects a position and/or a movement and/or an orientation of the endoscope and generates a correlation display having a display of the intracorporeal recording and a display of a view of the 3D recording data, in which the intracorporeal recording and the view of the 3D recording data with respect to the endoscope are correlated with one another.

### CROSS-REFERENCE TO RELATED APPLICATION(S)

This application is the United States national stage entry of International Application No. PCT/EP2022/052730, filed on Feb. 4, 2022, and claims priority to German Application No. 10 2021 103 411.6, filed on Feb. 12, 2021. The contents of International Application No. PCT/EP2022/052730 and German Application No. 10 2021 103 411.6 are incorporated by reference herein in their entireties.

### FIELD

The present disclosure relates to a surgical assistance system for use in a surgical intervention on a patient comprising a display device and an endoscope. In addition, the disclosure relates to an image display method, a computer readable storage medium, and a sterile space.

### BACKGROUND

In minimally invasive surgical interventions on soft tissue, such as abdominal surgery, surgical assistance systems with (only) computer-assisted navigation according to the prior art do not yet work precisely enough. Due to intraoperative movement of the soft tissue and the lack of a hard tissue reference, preoperatively acquired three-dimensional 3D image data, such as computed tomography (CT) image data or magnetic resonance imaging (MRI) image data, cannot be linked precisely enough to the patient during the surgical intervention. Therefore, surgeons still rely in particular on endoscopic images to find a target surgical field, to identify a tissue and to perform a treatment accordingly.

However, endoscopic images on their own are limited in terms of information content and in particular do not allow path planning or tissue identification based on preoperative (three-dimensional) 3D image data. This deficiency may lead to unintended damage to critical anatomical structures, such as nerves or blood vessels, as well as to under-resection or over-resection of the targeted pathological tissue. For the organ of the liver, for example, special mathematical models have been developed in order to correct intraoperative deformations based on surface extraction from the endoscopic image. However, these technologies are not reliable enough and do not guarantee the required high accuracy.

In general, surgeons benefit from having both intraoperative/intracorporeal endoscopic images and preoperative 3D images at their disposal. Currently, however, it is not possible to combine these two modalities of images without additional complexity while maintaining a high accuracy requirement.

Surgical assistance systems for endoscopic soft tissue navigation are known from the prior art, in which two different displays of two images are shown side by side or superimposed on one display. In addition to a first endoscopic intracorporeal image in a first partial area of the display, a three-dimensional virtual CT image is statically displayed in a second partial area of the display. However, in the surgical assistance system according to the prior art, an operator has to manually change the virtual CT image in order to readjust and adapt the display of the CT image to the current view of the endoscope with the intracorporeal image. This procedure ties up capacities, is highly demanding, is tiring and does not guarantee the necessary accuracy and thus safety during a surgical intervention.

Furthermore, navigation-based endoscopy is known from the prior art, in which an endoscope and a patient are followed/tracked via a camera. Marking elements/trackers are attached to both the endoscope and the patient for this purpose, with which registration is performed. In this way, CT image data can be correlated with the endoscopic intracorporeal image. However, such a system and method require a very high additional effort.

### SUMMARY

It is therefore the object of the present disclosure to avoid or at least reduce the disadvantages of the prior art and in particular to provide a surgical assistance system, an (image) display method, a storage medium as well as a sterile space, which provides OP participants at one glance, in particular a (leading) surgeon, with an intuitive and supportive fusion of information of an intracorporeal image of an endoscope as well as digital 3D image data of the patient, improves hand-eye coordination as well as ensures a safe access to the surgical field, in particular to avoid tissue damage by careless and unintentional actions as well as to minimize a duration of a surgical intervention. In particular, an OR participant is intended to benefit from both modalities, i.e. local navigation through the endoscope as well as global navigation through the patient's image data, in a simple way and be able to perform a surgical intervention intuitively and without tiring.

The surgical assistance system provides an OR participant, such as a surgeon, with both (image) modalities intraoperatively in an information-adequate and coordinated manner by correlating the two (image) modalities with each other and allowing the surgeon to use the endoscopic intracorporeal images for local guidance and the (views of the) 3D image data, in particular preoperative images, for global guidance as well as for further information on, for example, a tissue.

In the surgical assistance system, the intracorporeal image of the endoscope is initially registered to the digital 3D image data of the patient's body via an ‘image-based’ registration by detecting and determining at least one characteristic landmark/orientation point (in the image direction) and/or anatomical orientation in the intracorporeal image. This landmark and/or orientation is also found in the (virtual/digital) 3D image data. By this at least one reference point (landmark) and/or orientation the intracorporeal image can be correlated (geometrically) with the 3D image data. In particular, several characteristic landmarks, preferably two characteristic landmarks, particularly preferably three characteristic landmarks, are detected and determined in the intracorporeal image.

In other words, two sides/spaces exist in the surgical assistance system: a first, real (geometric) space, with the real endoscope and the real body of the patient, and a second, virtual (geometric) space, with a virtual endoscope and a virtual body (region) of the patient. The aim is to ‘link’, i.e. correlate, the real space with the virtual space in a simple way, so that the geometric relation of recording head, in particular endoscope, to body in the real space corresponds to the geometric relation of recording head, in particular endoscope, to body in the virtual space. The assumption is made that the digital 3D image data, at least in part, corresponds geometrically seen to the real body of the patient and approximately represents a 1:1 image in the virtual space.

If, during a surgical intervention, the endoscope is inserted into the patient's body, manually or robotically guided, this endoscope detects an intracorporeal area of the body via its recording head, in particular optically, and produces an intracorporeal image, such as a two-dimensional or three-dimensional image. This captured image or the captured anatomical structure is found both in the real body and in the virtual, digital body, as explained above. Now, in order to perform an initial registration between the real endoscope and the real patient (or body of the patient), a registration between a virtual endoscope, or at least a virtual recording head, and the virtual body of the patient is performed via the intracorporeal image, so to speak, and is ultimately ‘transferred’ to the real space. According to this, the real space and the virtual space are, so to speak, linked to each other and the virtual space can in a sense be regarded as an ‘image’ of the real space.

The registration can be performed in particular via the following different registration methods:
point to point registration, wherein at least three points are required for unambiguous pose recognition. Here, at least three spaced (anatomical) points are detected as landmarks in the intracorporeal image (these span a plane). This detection can be performed automatically and/or with the help of a manual input. In particular, the control unit may then be adapted to detect or respectively determine three corresponding points/landmarks in the 3D image data and to perform the registration accordingly;
point and orientation registration, wherein at least one point as landmark and one orientation are required. In this case, a bijective definition is achieved via a reference landmark and an orientation; the term orientation means in this case, in particular with respect to a reference coordinate system, that three angles are specified for a definition of the orientation, so that the orientation is unambiguous.
surface matching, wherein a three-dimensionally detected surface (3D surface) of the intracorporeal image (with a plurality of landmarks approximating the 3D surface) is correlated with a corresponding 3D structure in the 3D image data, such as a segmented (three-dimensional) structure, and is thus initially registered. For example, a (partial) surface of a liver in real space can be correlated with a segmented 3D structure of the liver in MRI image data.

Via the initial registration via the at least one landmark and/or orientation, the relation of intracorporeal images to the 3D image data (with respect to at least the recording head) is available at least initially.

According to this, the tracking system detects a movement (variable pose over time) of the real endoscope, in particular the recording head, in real space. The movement or the endoscope movement data may have translations (transformations) and/or rotations, which can add up to six degrees of freedom. In particular, the transformation or a transformation matrix of a handling portion, in particular the handpiece, of the endoscope to its recording head is known or determinable, so that starting from a movement of the handling portion, a movement of the recording head as well as vice versa is determinable, in particular by the control unit. Further preferred, a transformation or transformation matrix from the recording head to the intracorporeal (endoscope) image and/or a transformation from the handling portion to the intracorporeal image can be known or determinable, so that from a movement of the intracorporeal image, a movement of the recording head or of the handling portion and vice versa is determinable, in particular by the control unit. In particular, translational movements in three directions and rotational movements about three axes can be provided as endoscope movement data to the control unit.

The control unit transfers the endoscope movement data of the real space to the 3D image data, in particular to the virtual recording head in the virtual space. It can also be said that an absolute correlation is carried out with the registration and a subsequent relative correlation with the detection of a movement.

It can also be said that the endoscope movement data change a transformation matrix between a local coordinate system of the recording head to a local coordinate system of the patient's body (3D image data) in virtual space and a view of the 3D image data is adapted accordingly. It should be noted that of the up to six recorded degrees of freedom of the endoscope movement data, in particular, only a selected amount can also be passed into the virtual space as endoscope movement data. A subset of endoscope movement data is useful, for example, if a position of the endoscope changes very little during an operation, but mainly only an orientation.

With the generated correlation display/correlation image/correlation view, the surgical assistance system provides the surgeon with both a local and global navigation modality and improves the surgeon's orientation. The surgeon is able to ‘see’ behind anatomical structures in the native image/intracorporeal image via the correlation display output by, for example, an OR monitor. The surgeon can better and more efficiently navigate in order to reach the desired region and knows where to operate. Also, safety during the surgical intervention is increased and surgical quality is improved. Improved orientation leads to shorter intervention times. In particular, the surgeon no longer has to superimpose sectional images in his/her head or estimate where he/she is at any given moment. This leads to a reduction in complications and thus to a reduction in additional costs and risks for the patient and the hospital.

In other words, the object is achieved in a surgical assistance system for use in a surgical intervention on a patient by said assistance system comprising: a display device, in particular an OR monitor, for displaying a visual content, in particular a picture, an endoscope, in particular a video endoscope, with a distal imaging recording head, which is provided and adapted to create an intracorporeal image of the patient and to provide said intracorporeal image in a digital/computer readable manner, a data-providing unit, in particular a storage unit which is provided and adapted to provide, in particular to prepare and provide, digital 3D image data, in particular preoperative 3D image data, of a patient in a digital/computer-readable manner, and a control unit/calculation unit/computer unit adapted to process the intracorporeal image of the endoscope and the 3D image data. The surgical assistance system further comprises: a registration unit, which is provided and adapted to detect, in the intracorporeal image of the endoscope, at least one, in particular several, preferably two, particularly preferably three, in particular predetermined, anatomical landmarks and/or an anatomical orientation, and to determine at least one corresponding anatomical landmark and/or an orientation in the provided 3D image data, and to register the intracorporeal image with the 3D image data, at least initially, via the corresponding anatomical landmarks and/or orientation and to register the endoscope, in particular the recording head, relative to the (body of the) patient; a tracking system, which is provided and adapted to constantly/continuously detect a pose of the endoscope and/or a movement of the endoscope, in particular of the recording head, and to provide the control unit with endoscope movement data. The control unit is further adapted to generate a correlation display with both (at least) a display of the intracorporeal image and (at least) a display of a view of the 3D image data, in which the intracorporeal image and the view(s) of the 3D image data are correlated with respect to the endoscope, in particular the recording head. Here, the detected endoscope movement data is transferred to at least one virtual position and/or orientation of a virtual recording head, in particular of a virtual endoscope, in the view of the 3D image data for a correlated movement, and the control unit is adapted to visually output the correlation display thus generated via the display device. The control unit may, for example, include a processor (CPU) for corresponding processing and control. The data-providing unit can also process the data if context-related changes of the model occur (e.g. by deformation or separation of tissue).

Basically, in a surgical assistance system, the present disclosure thus provides a first (real) image, i.e. the intracorporeal image through the endoscope, and a second (virtual) image in the form of a (predetermined) view of 3D image data, such as CT image data or MRI image data. Both images are visually displayed to an OR participant such as the surgeon on in particular an OR monitor. In particular, the current, real endoscope video image is displayed as an intracorporeal image in one (partial) area of the correlation display and thus of the OR monitor, while a (selected) correlated view of the 3D image data is displayed in another (partial) area of the OR monitor. In particular, both displays are arranged side by side. Alternatively, both displays can be overlaid (augmented reality). This enables a surgeon to perform global navigation via the 3D image data view in order to reach a target surgical area, while the endoscopic intracorporeal image provides detailed images of approximately the actual, current pose of a soft tissue. Since both images are correlated to each other, the surgeon does not have to make any manual adjustments or virtual rotations or shifts of the images relative to each other. In particular, the correlation of the two images is automated by the surgical assistance device.

In yet other words, the present disclosure relates to an intraoperative surgical assistance system which displays an endoscopic picture (intracorporeal image) and a, in particular preoperative, 3D picture (3D image data) of the patient, in particular side by side. Both pictures (images) are correlated and relate to the current position and/or orientation of the recording head, in particular of the endoscope. Preferably, when the endoscope is inserted into a surgical field, in particular predefined anatomical landmarks and/or orientations are selected by the surgeon based on the endoscopic image (intracorporeal image) and correlated with corresponding anatomical landmarks and/or orientations in the 3D image data, wherein this step is referred to as registration. Different registration methods are possible:
point to point, at least 3 points
point and orientation
surface matching, correlating the 3D surface from the endoscope image (intracorporeal image) with a, in particular corresponding segmented, structure in the 3D picture (3D image data).

Although registration via a landmark has the advantage that a distance to an object, such as a liver, is also captured and the absolute dimensions of intracorporeal image and 3D image data can thus be ‘matched’, however, it may also be sufficient if only the object is rotated correctly (only angle adjustment). This can be useful for segmented displays, in particular, since the 3D image data may include only one organ.

The term 3D defines that the image data of the patient is available spatially, i.e. three-dimensionally. The patient's body or at least a partial area of the body with spatial dimensions may be digitally available as image data in a three-dimensional space with a Cartesian coordinate system (X, Y, Z), for example.

The term pose defines both a position and an orientation. A position can be specified in a Cartesian coordinate system with (X, Y, Z) and the orientation with three angles (α, β, γ), for example.

The term view describes, in particular in the 3D image data, in particular at a (predeterminable) point a (predeterminable) viewing direction/perspective with in particular (predeterminable) rotation, similar to a selectable view in a CAD model. In a way, a pose of a viewpoint is defined.

The term real defines the ‘side’ of the physical (real) endoscope and patient on which the actual intervention is performed, whereas virtual defines the ‘side’ of the digital, virtual endoscope and the captured virtual patient data, which are available in a computer-readable form and can be digitally manipulated in a simple manner, for example to display individual information.

Advantageous embodiments are explained below.

In a preferred embodiment, the tracking system can be configured in particular as an endoscope-internal tracking system and the endoscope, in particular a handling portion and/or the recording head, can have an internal movement sensor, preferably an acceleration sensor and/or a rotation rate sensor, in particular an inertial measurement unit (IMU; inertial measurement unit), for detecting the endoscope movement data. Thus, the surgical assistance system does not require any further external systems or measuring devices, but only a sensor integrated in the endoscope, which is spatially small, is sufficient. Via the internal movement sensor, a movement of the endoscope, in particular of the recording head, can be detected autonomously and can be provided digitally as endoscope movement data to the control unit. In other words, in order to determine the movement of the endoscope, in particular of the recording head, the endoscope can have at least one movement sensor, preferably internal sensors such as in particular an IMU.

According to a further embodiment, external sensors or reference marks that are detected by a camera system can be provided for the tracking system as an alternative or in addition to endoscope-internal sensors. In particular, the tracking system can have a camera system that (continuously) detects the endoscope spatially, in particular a handling portion, determines its pose and provides this to the control unit as endoscope movement data. Hereby, the pose of the recording head can be inferred by a predetermined geometric relation. In other words, the tracking system can also have an external camera, in particular a 3D camera, which detects an intervention region and, in the case of a rigid endoscope, continuously detects a pose of the handling portion and, on the basis of the detected handling portion, temporally determines the pose and thus the movement of the endoscope, in particular of the recording head.

According to a preferred embodiment, for forming a tracking system, the control unit can be adapted to determine the movement of the endoscope via image analysis/data evaluation of moving anatomical structures in the intracorporeal image and to provide this as endoscope movement data. Using suitable image analysis methods, a change in the picture can be used to infer a movement. This can be compared with a video recording of a front camera of a car, for example, where a movement is detected on the basis of road markings or a moving horizon. The tracking system in the form of the adapted control unit thus calculates a movement, in particular of the recording head, on the basis of the time-varying intracorporeal image. In particular, the optical parameters of the endoscopic intracorporeal image are known and/or the recording head, in particular the endoscopic camera of the recording head, is calibrated. In other words, movements of the endoscope can also be detected via the endoscopic picture/intracorporeal image by analyzing in which direction and in particular at which speed the (anatomical) structures in the picture are moving. One advantage of such an image analysis is that even without movement sensors, the endoscope movement data can be determined.

In particular, the tracking system can comprise on the one hand the movement sensor provided in the endoscope, in particular the acceleration sensor and/or rotation rate sensor, particularly preferably the inertial measurement unit, and on the other hand the control unit can be adapted to detect the endoscope movement data via the image analysis, wherein a combined value of the two results, in particular an adjusted mean value or a weighted value of the endoscope movement data, is calculated from the at least two results of the endoscope movement data as endoscope movement data (as final result). The weighting can be done for each individual element/component of rotation and translation (six elements/degrees of freedom). For example, inertial measurement units (IMU) can determine rotations more accurately than translations. In particular, the rotation data is provided from the at least two results via the inertial measurement unit (IMU) and the translation data is provided via the image analysis for the endoscope movement data. In particular, the weighting may be such that in the result of the IMU, the rotation components are weighted with more than 80%, in particular 100%, whereas the translation components are weighted with less than 20%, in particular 0%, wherein in the result of the image analysis the rotation components are complementarily weighted with less than 20%, in particular 0%, and the translation components are weighted with more than 80%, in particular 100%. The mean value can be, for example, an arithmetic mean value, a geometric mean value or a quadratic mean value, which is formed in particular for each individual component. In this way, (at least) two different ‘sensor systems’ are combined. Redundant detection with combination, in particular averaging or weighting, can further minimize system-related inaccuracy errors and increase an accuracy. Thus, in particular, the internal sensors and image analysis can be combined to determine the movement of the endoscope. As a result, the surgeon can track the actual position of the endoscope, in particular of the recording head, in the 3D pictures/3D image data and thus identify critical structures or follow a path from a preoperative plan.

According to a further preferred embodiment, the control unit can be adapted, in particular on the basis of a database stored in the storage unit with geometrically predefined structures of medical instruments, to recognize at least one (stored) medical instrument in the intracorporeal image in a correct pose and to display the recognized medical instrument in the view of the 3D image data as a virtual medical instrument in a correct pose, in particular correlated, so that the medical instrument is displayed in both images. The term correct pose defines the combination of correct position and correct orientation, i.e. at the matching position and pose. In other words, in particular, at least one instrument can be displayed in both images, so that the surgeon can visualize not only the position and/or orientation of the endoscope in the 3D image data, but also the position and/or orientation of the medical instrument. For this purpose, the medical instruments are detected in the intracorporeal image and virtually superimposed on their (actual) position in the 3D image data (3D picture). The prerequisite for this is that the instruments are visible in the endoscopic picture and their geometric shape is known.

In particular, data on the recording head, in particular a camera head type, and/or data on the endoscope, in particular an integrated endoscope and/or a type of ‘dummy endoscope’ for a camera head, can also be stored in a database stored in the storage unit. For example, stored geometric data of the recording head can be displayed in the correlation display accordingly.

In particular, the control unit can be adapted to determine a cone of view of the recording head on the basis of the position and/or orientation of the endoscope, in particular of the recording head, the data stored in the storage unit about the endoscope, in particular about optical parameters of the recording head, and preferably a distance from the targeted anatomical structures, and to include a virtual cone of view of the virtual recording head in the correlation display in the display of the 3D image data. In this way, the user can see which area he is currently viewing on the 3D image data. This is advantageous, for example, if the scales are not the same and the 3D image data is zoomed out. The term cone of view describes a three-dimensional volume formed by a field of view defined by an optical system and a corresponding distance to a front.

Preferably, the registration unit can perform a re-registration beyond the initial registration at at least one further, in particular predetermined, point in time in order to further increase the accuracy of the correlation. Since, starting from a registered (absolute) start position of the recording head, the endoscope movement data is relative data (without external absolute reference), a re-registration (as absolute correlation) can further increase the accuracy. In particular, the subsequent registration can be performed after a predetermined time or after a predetermined sum of movements. In particular, multiple repeated registrations can also be performed after a predetermined time, a predetermined distance, or a predetermined sum of movements as a ‘reset’ in each case. Thus, a re-registration between the intracorporeal image of the endoscope and the 3D image data (3D model/3D picture) can be performed at any time to further improve the accuracy in case of soft tissue movements.

In a preferred embodiment, the endoscope may be configured as a rigid video endoscope. In a rigid endoscope, there is a constant geometric relation between a handling portion and the recording head. For example, a movement sensor can be provided in a handpiece to detect the movement in the handpiece and the control unit can be adapted to infer the movement of the recording head based on the detected movement data and the geometric relation.

Preferably, the control unit can be adapted to (virtually) display in the correlation display in the display of the view of the 3D image data and/or of the display of the intracorporeal image, in addition to the real and/or virtual endoscope, pre-planned medical instruments and/or implants, in particular a pre-planned trocar, stored in a storage unit in the correct pose. In particular, a pre-planned trocar can be displayed in the 3D image data or in the view of the 3D image data relative to the current position of the endoscope.

According to a further preferred embodiment, the control unit may be adapted to display a planned path/trajectory in the correlation display in the display of the view of the 3D image data and/or of the intracorporeal image in order to guide a surgeon to the surgical field. This further improves an orientation of the surgeon. In particular, a trajectory can be indicated by a superimposed arrow with arrowhead in the direction of the planned path, which adjusts as the endoscope moves and always points in the direction of the path. Alternatively or additionally, a planned path can also be displayed in the 3D image data in the form of a three-dimensional line along the path. Alternatively or additionally, pre-operatively planned annotation points can also be superimposed intraoperatively in the 3D image data, for example to approach a target area or to recognize critical structures.

Preferably, the control unit can be adapted to generate the view of the 3D image data as 3D scene/3D rendering and/or as two-dimensional cross-sections relative to a picture-coordinate system of the endoscope and/or along an image axis of the endoscope, in particular of the recording head, and/or as a virtual endoscope image. The 3D image data can thus be, for example, a rendered 3D model, with or without segmented anatomical structures, which can be freely moved in the virtual space and displayed from different positions and views from different viewing angles with, in particular, different transparencies of different anatomical structures, similar to a CAD model. The control unit then creates a selected view of the 3D scene. Also, for example, sets of anatomical structures in the virtual space can be hidden to obtain an even better view of the relevant structures. Alternatively or additionally, the 3D image data may also be available as (segmented) two-dimensional cross-sections relative to a coordinate system of the endoscope, in particular a picture-coordinate system of the endoscope, and/or along an axis, such as in the case of MRI or CT image data. The picture-coordinate system corresponds to the (local) coordinate system of the intracorporeal image. Alternatively, two-dimensional cross sections can be created relative to a local coordinate system of the recording head. The picture-coordinate system of the intracorporeal image can also be calculated back via a transformation to the local coordinate system of the recording head and further via a transformation to a local coordinate system of a handpiece. The control unit can then, for example, similar to ultrasound images, create the view of the 3D image data along the axis of the image direction or recording head, in particular of the endoscope, corresponding to the position of the recording head, in particular of the endoscope. This allows, for example, to detect anatomical structures behind the picture captured by the endoscope. Alternatively or in addition to the cross-sections along the image axis, diagonal or individual cross-sectional displays can also be created, that are predefined or depend for example on the anatomy. Preferably, X sections and/or Y sections and/or Z sections viewed with respect to a reference coordinate system of the endoscope (with X-Y-Z axes) can be created. In particular, for Z sections (i.e., the sections parallel to the field of view of the endoscope and perpendicular to a local Z axis of the reference coordinate system of the endoscope), the section can be shifted accordingly via a back and forth movement of the endoscope. A range of movement toward an object may be limited, for example, by a working distance. In a rigid endoscope, a Z-axis is in particular coaxial to its longitudinal axis and the Z-sections are accordingly arranged orthogonal to the Z axis and thus parallel to the field of view. Alternatively or additionally, the control unit can create the view of the 3D image data as a virtual endoscope picture/endoscope perspective that imitates the real image of the recording head. In other words, a view of the 3D image data is created in which the virtual recording head of the virtual endoscope has the same pose (i.e. position and orientation) as the real recording head, and a virtual image is created with exactly the same optical parameters as the real endoscope, so that the real intracorporeal image is displayed as a virtual intracorporeal image in virtual space (1:1 display real image-virtual image). In this case, the surgeon ‘sees’ both in the real and the virtual displays in exactly ‘the same direction at the same position’. In other words, the 3D image data, in particular the view of the 3D image data (3D picture) can be displayed as a 3D scene/3D rendering, with or without segmented anatomical structures. Alternatively or additionally, the 3D picture can be presented as 2D cross-sections along the axis of the endoscope, similar to ultrasound images. Alternatively or additionally, the 3D pictures can be displayed as a virtual endoscopic picture that mimics the viewing direction of the endoscope, similar to a fly-through model.

Preferably, the surgical assistance system has a display selection unit in which a predefined number of different views are stored. In particular, a bird's eye view, an endoscope view (in the image direction), a side view and a bottom view are stored. When a view is selected, in particular by the surgeon, the control unit adjusts the view of the 3D image data to the selected view accordingly. In other words, the surgical assistance system may have pre-configured settings that allow the surgeon to adopt different views of the 3D image data (displays of the 3D picture) such as bird's eye view, endoscope view, side views, floor view, for the region to be examined, to adopt different perspectives and to take into account the individually available and different contents for a surgeon's decisions.

In particular, the control unit performs the function of the tracking system.

According to an embodiment, the surgical assistance system may further comprise a robot and the endoscope, in particular a handling portion of the endoscope, may be robot-guided, in particular via a robot arm, and the tracking system may determine the pose and/or orientation and/or movement of the endoscope, in particular of the recording head, via an associated pose and/or orientation and/or movement of the robot, in particular of the robot arm. In the case of a robot-assisted surgical intervention in which the endoscope is guided via the robot, preferably a position and/or orientation and, seen over time, thus a movement of the endoscope, in particular of the recording head, can be calculated directly from an axis system or control commands with corresponding transformation. The detection of the position and/or orientation and/or movement thus does not take place in the endoscope itself, but in the robot. In other words, in a robotic system, the movement and/or position and/or orientation of the endoscope is derived from the robot, in particular the robot arm.

With regard to an image display method for correlated display of two different images, the objects and objectives of the present disclosure are solved by the steps of: reading in digital 3D image data, in particular pre-operative 3D image data, of a patient; creating an intracorporeal image through an endoscope; detecting at least one landmark and/or orientation in the intracorporeal image; determining a corresponding landmark and/or orientation in the 3D image data; registering the 3D image data to the intracorporeal image via the at least one detected landmark and/or orientation; generating at least one correlation display with a display of the intracorporeal image and a display of a view of the 3D image data and preferably outputting the correlation display by the display device; continuously detecting a pose and/or orientation and/or a movement of the endoscope, in particular of the recording head; transferring the detected movement of the endoscope, in particular of the recording head, to at least one virtual position and/or orientation of a virtual recording head, in particular of a virtual endoscope, in the view of the 3D image data and generating an updated correlation display with the intracorporeal image and the updated view of the 3D image data and outputting the correlation display by the display device.

According to an embodiment, the image display method may preferably comprise the following steps: detecting 3D image (data), in particular preoperative 3D image (data); preferably segmenting 3D image (data); providing the 3D image data, in particular the segmented image, in a surgical assistance system; preferably handling endoscope and/or creating an intracorporeal image; targeting and detecting anatomical landmarks in the native endoscopic intracorporeal image; determining and detecting corresponding landmarks in the 3D image data; registering the two images (the intracorporeal image and the 3D image data) via the detected landmarks; (detecting and) determining a movement of the recording head, in particular of the endoscope, in particular by using at least one endoscope internal sensor and/or a picture analysis/image analysis of the endoscopic intracorporeal image; transferring the detected movement to the 3D image; and correlated output of the two images, in particular side by side and/or superimposed.

Preferably, the display method has a further step of re-registration.

According to a further preferred embodiment, the display method may comprise the steps of: detecting a medical instrument and its pose in the intracorporeal image; pose-correct virtual overlay display/display of the detected instrument in the 3D image data;

Preferably, the display method may comprise the step of: pose-correctly displaying the virtual endoscope, in particular the virtual recording head in the 3D image data.

According to a further preferred embodiment, the display method may comprise the step of: superimposing a pre-planned medical instrument, in particular a pre-planned trocar, on the intracorporeal image and/or the 3D image data.

In particular, the display method may comprise the steps of: detecting an input of a view selection, in particular a selection from a set of a bird's eye view, an endoscope view, a side view, and a ground view; applying the view selection to the view of the 3D image data such that in the correlation display the selected view is displayed.

Preferably, the display method may further comprise the steps of: determining, based on the detected landmarks and the determined corresponding landmarks, a deformation of a real anatomical structure, in particular a deformation relative to an initial anatomical structure; and transferring this determined deformation to the virtual anatomical structure in the 3D image data in order to adjust the 3D image data accordingly. Thus, a deformation can be determined based on corresponding landmarks and their relative change, in particular to initial landmarks, and this deformation can then be transferred to the 3D image data accordingly in order to correct it. Especially in case of a registration via surface registration this method has advantages, where not only a rigid correlation with the 3D image data is made, but a segmented object, such as a liver, is deformed accordingly in the 3D image data, so that the 3D image data fits to the real 3D structures captured by the endoscope. These steps can be performed in particular by the control unit of the assistance system.

At this point, it is noted that features of the display method of the present disclosure are transferable to both the surgical assistance system of the present disclosure and vice versa.

With respect to a computer-readable storage medium, the objects and objectives of the present disclosure are solved by the computer-readable storage medium comprising instructions which, when executed by a computer, cause the computer to perform the method steps of the image display method according to the present disclosure.

With respect to a generic sterile space, the object of the present disclosure is solved in that the medical sterile space comprises a surgical assistance system according to the present disclosure.

### BRIEF DESCRIPTION OF THE DRAWINGS

The present disclosure is explained below with reference to preferred embodiments with the aid of accompanying figures.

FIG. 1 shows a schematic view of a surgical assistance system of a first preferred embodiment;

FIG. 2 shows a schematic partial view of a rigid endoscope of a surgical assistance system of a further preferred embodiment with associated intracorporeal image, whose movements are transferred to a virtual endoscope with a view of the 3D image data;

FIG. 3 shows a schematic view of an image-based registration of a surgical assistance system;

FIG. 4 shows a schematic perspective view of a video endoscope used in a surgical assistance system of a preferred embodiment;

FIG. 5 shows schematic view of different perspectives for a view of the 3D image data; and

FIG. 6 shows flowchart of an image display method of a preferred embodiment.

The Figures are merely schematic in nature and are only intended to aid understanding of the disclosure. Identical elements are provided with the same reference signs. The features of the various embodiments may be interchanged.

### DETAILED DESCRIPTION

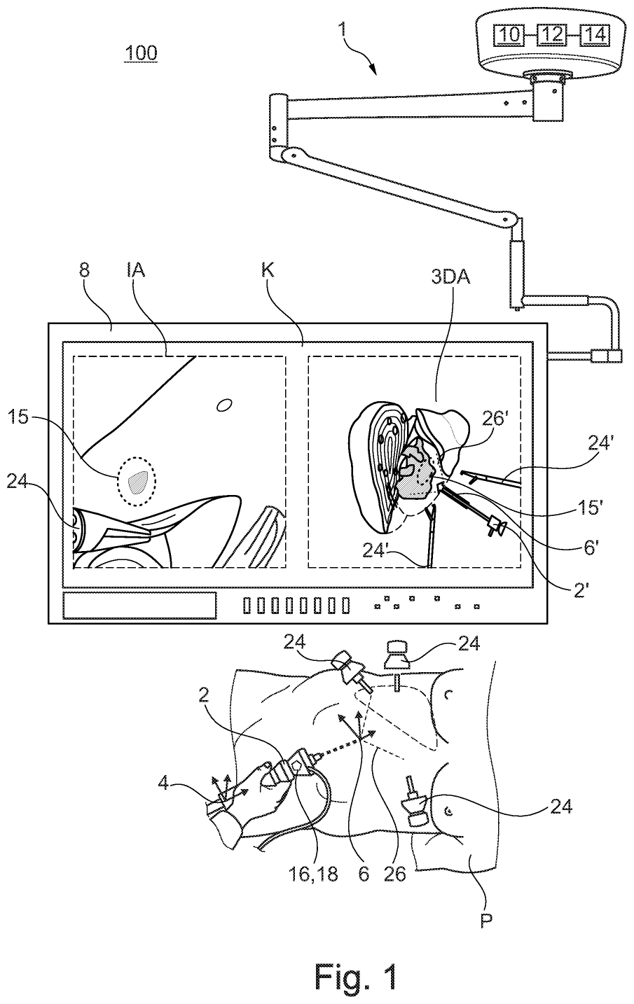

FIG. 1 shows a schematic view of a surgical assistance system 1 (hereinafter referred to only as assistance system) of a first preferred embodiment for use. The assistance system 1 is used in a medical sterile space in the form of an operation room 100 of a preferred embodiment in order to support OR participants, in particular a surgeon, via suitable visualization during a surgical intervention on a patient P (shown here only schematically). In a central, sterile, surgical intervention region, a minimally invasive intervention is performed on the patient. A rigid video endoscope 2 (hereinafter referred to only as endoscope) with a handpiece/handle 4 and a spaced frontal imaging recording head 6 is positioned on the endoscope-shaft side into the body interior of the patient. The recording head has an endoscope video camera (not shown) for imaging, which creates a two-dimensional intracorporeal image IA of the body interior of the patient P as a current (live video) image in the direction of a longitudinal axis of the endoscope 2 on the front side via a CMOS sensor and provides it digitally. Via a display device in the form of an OR monitor 8, a visual content for OR participants, such as a display of the intracorporeal image IA, can then be reproduced. It is noted that the endoscope 2 can alternatively be guided at its handling portion by a robotic arm.

Furthermore, the surgical assistance system 1 has a data-providing unit in the form of a storage unit 10 in which digital, preoperative 3D image data 3DA of the patient P to be treated are stored in digital/computer-readable form. 3D image data 3DA can be (segmented) MRI image data or CT image data, for example, which virtually or digitally depict a body of the patient P or at least a part of the body.

For detection, processing and calculation as well as for control, the assistance system 1 has a control unit 12, which is adapted to process the intracorporeal image IA of the endoscope 2 as well as the 3D image data, and to control the OR monitor 8 accordingly.

Furthermore, the surgical assistance system comprises a registration unit 14, which in this embodiment is configured as a specially adapted control unit 12. Specifically, the control unit 12 is adapted to detect anatomical landmarks 15 in the intracorporeal image IA of the endoscope 2 and to determine corresponding anatomical landmarks 15′ in the provided 3D image data. In the intracorporeal image IA of the endoscope 2, which is inserted into the body of the patient P and captures a targeted anatomical structure as a two-dimensional picture, the surgeon selects three predefined characteristic (real) landmarks 15 in this embodiment. These three landmarks 15 in real space are also found as three (virtual) landmarks 15′ in virtual space. The intracorporeal image IA is initially correlated, i.e. registered, with the 3D image data via the total of two times three corresponding anatomical landmarks 15, 15′. Also, the endoscope 2, in particular the recording head 6, is registered relative to the patient P in this way. Thus, a geometric relation between the virtual recording head 6′ and the virtual body of patient P (3D image data) is established, which corresponds to the real geometric relation between the real recording head 6 and the real body of patient P. In the virtual space, the control unit 12 calculates a corresponding transformation matrix and transfers it to the real space. Thus, the pose of the recording head 6 of the real endoscope 2 relative to the body of the patient P is determined. In other words, the (real) intracorporeal image IA is initially correlated, i.e. registered, with the (virtual or digital) 3D image data 3DA via at least one characteristic landmark 15, 15′, which occurs in both images, in order to register or determine the pose of the (real) endoscope 2, in particular of the recording head 6, relative to the patient P.

In particular, the control unit 12 may be adapted to determine, knowing optical parameters of the recording head 6 or a distance from the recording head 6 to the anatomical landmarks, such a view of the 3D image data 3DA in which these three landmarks have a substantially equal distance to each other in the 3D image data 3DA as in the intracorporeal image IA.

The surgical assistance system 1 also has a tracking system 14 that continuously detects a movement of the endoscope 2 and of the recording head 6 and provides it to the control unit 12 as endoscope movement data. In this embodiment, a combination of an endoscope internal sensor and an image analysis performed by the control unit 12 is used.

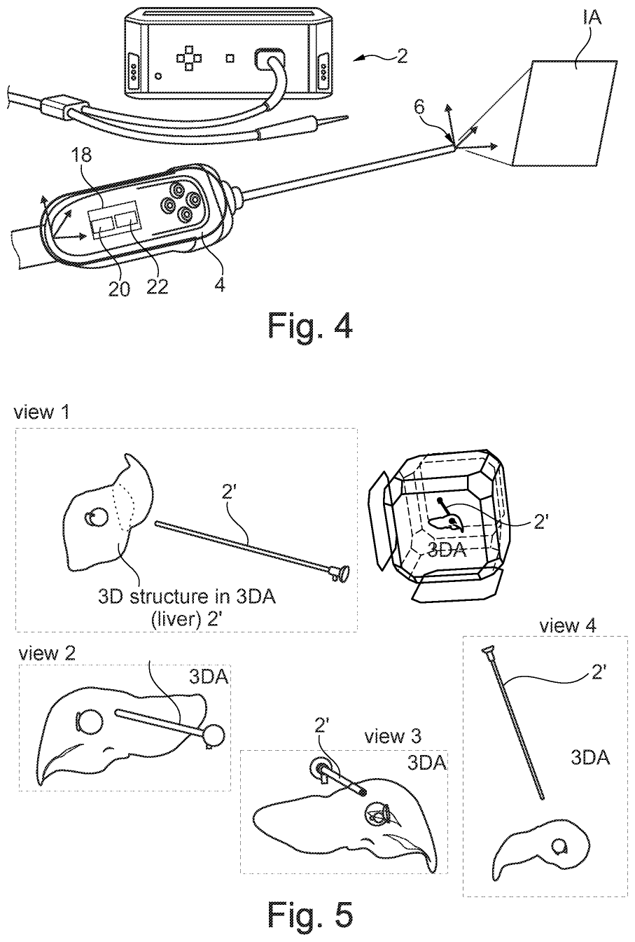

Specifically, the handle 4 of the rigid endoscope 2 has an inertial measurement unit (IMU) 18 with multiple inertial sensors, in this case an acceleration sensor 20 and a rotation rate sensor 21 (three-axis acceleration sensor and three-axis gyroscope) for an inertial navigation system design (see also FIG. 4). The respective accelerations in the three directions result in a total acceleration in one direction in real space (vector). The detected rotation rates around the three axes can be mapped, for example, as a rotation matrix. Thus, a movement of the endoscope in real space is detected continuously (due to technical limitations approximated in discrete time intervals with time intervals as small as possible) and passed on to the control unit 12 as endoscope movement data with translations and rotations. The IMU 18 detects not only the movement of handle 4 but also that of the recording head 6 via the fixed geometric relationship between handle 4 and recording head 6.

Furthermore, in the surgical assistance system 1, the control unit 12 is adapted to perform an image analysis based on the intracorporeal image IA. The control unit 12 performs data processing to the effect that it detects a temporal change in the anatomical structures and thus determines a movement of the structures (in particular a change in direction) and, on the basis of these changes, a movement of the recording head 6 and thus of the endoscope 2. Thus, in addition to sensory endoscope movement data, image-analytical endoscope movement data is also available, which can be linked together to minimize measurement errors and increase accuracy. An average value of the two endoscope movement data is formed and provided as a result to the control unit 12.

Since the control unit 12 is additionally adapted to generate a correlation display K with a juxtapositioning of both a display of the intracorporeal image IA and a display of a view of the 3D image data 3DA, in which the intracorporeal image IA and the view of the 3D image data 3DA are correlated to each other with respect to the endoscope 2, in particular to the recording head 6. For this purpose, the detected endoscope movement data are transferred to at least one virtual position and/or orientation of a virtual recording head 6′, in particular of a virtual endoscope 2′, in the view of the 3D image data 3DA for a correlated movement. In particular, the virtual recording head 4′ is moved in the same way as the real recording head 4. A view of the 3D image data is adapted accordingly. Finally, the control unit 12 is adapted to visually output the correlation display K thus generated via the OR monitor 8. The correlation display K is a juxtapositioning of the endoscopic intracorporeal image IA and the preoperative 3D image data, wherein both images are correlated with respect to the movement of the endoscope.

The view of the 3D image data allows a global orientation, while the endoscopic intracorporeal image allows a precise spatial orientation. The surgical assistance system 1 of the present disclosure is adapted to display both modalities side by side and in a correlated manner. This allows the surgeon to better perform the operation and directly use the 3D image data with in particular preoperative information, despite the fact that due to the intraoperative deformation of the tissue, the endoscopic intracorporeal image and the 3D image data do not always perfectly match. For precise intervention, he/she can furthermore rely on the endoscopic intracorporeal image. The correlated 3D image data or 3D pictures enable the surgeon to avoid critical structural damage, to better find his surgical target, and to execute a preoperative plan precisely and efficiently. By global guidance with the 3D image data, the assistance system 1 can also shorten a learning curve when using endoscopic intracorporeal images. In particular, orienting a position and/or orientation of the endoscope, in particular the recording head, to the 3D image data or to the segmented 3D model, respectively, leads to an improved orientation of the surgeon in the region of interest.

Thus, after an initial registration, in the surgical assistance system 1 of the present disclosure, the transformation matrix in virtual space can be changed, so to speak, to the same extent as the transformation matrix in real space, in order to entrain the endoscope 2′, in particular the recording head 6′, in virtual space equal to the endoscope 2 or the recording head 6, respectively, in real space and to adapt (correlate) the view of the 3D image data 3DA accordingly, in particular to entrain it.

An initial registration (as an absolute correlation) via the registration unit 14 can be performed in particular as follows. A local recording head coordinate system can be assigned to the recording head 6 of the endoscope 2, in the real space. A local coordinate system can also be assigned to the body of the patient P. For an initial registration, a transformation matrix between these two local coordinate systems has to be calculated and provided in order to determine an initial pose (position and orientation) of the recording head and thus in particular of the endoscope relative to the body of the patient P via this transformation matrix. Also in the virtual space, a local recording head coordinate system can be assigned to the virtual recording head 6′ as well as a local coordinate system to the ‘virtual body’ of the patient. One could also say that a virtual body or at least a partial area, such as an organ, is congruently assigned to the real body. This establishes a basic link between the real space and the virtual space. This is clearly understandable, since in particular preoperative 3D image data, such as CT image data or MRI image data of the entire body or at least of a partial area of the body are captured, which represent the real body 1:1 in space. The real intracorporeal image IA of an anatomical structure with the at least one detected landmark is also found in the virtual 3D image data and enables a registration, in particular between the virtual recording head 6′ and the virtual body, via a matching process. Hereby, an initial transformation matrix is obtained in the virtual space, which can be ‘transferred’ to the real space to perform an initial registration. Consequently, after registration in both spaces, at least the geometric pose of the recording head is correlated to the patient's body and thus also the intracorporeal image to the 3D image data.

With the aid of the surgical assistance system 1 of the present disclosure, an intracorporeal image IA with a view of the 3D image data 3DA can be generated with only a very slight modification of the hardware of existing endoscopes, i.e. by supplementing a (standard) endoscope with an internal (movement) sensor 18 and/or by detecting the movement of the endoscope 2 via an external camera such as a 3D stereo camera, as well as by appropriate adaptation of a control unit, which in particular can be carried out via an appropriately stored program, without additional space-intensive and error-prone hardware devices, an intracorporeal image IA can be correlated with a view of the 3D image data 3DA and output via the OR monitor 8. This assistance system 1 improves an orientation of a surgeon and thus also safety of a patient P.

The control unit 12 is also adapted via a database stored in the storage unit 10 with geometrically predefined structures of medical instruments 24 in the intracorporeal image IA to detect such a medical instrument 24 and also to detect an associated pose in real space. This detected medical instrument 24 is then virtually superimposed as a virtual medical instrument 24′ in the 3D image data 3DA in its detected pose via its stored geometric structures, so that the medical instrument 24, 24′ is displayed in both displays of the images IA, 3DA. In this way, an orientation can be further improved.

In addition to the detection of medical instruments 24 in real space, the control unit 12 is also adapted to display pre-planned virtual medical instruments 24′, in particular a pre-planned trocar, in the view of the 3D image data (3DA) (superimposed) in virtual space. These pre-planned virtual medical instruments 24′ including their pose relative to the (virtual) body of the patient are also stored in the storage unit 10.

In addition, a planned path to the surgical field is stored in the storage unit 10, which can be displayed accordingly in the 3D image data, for example as an arrow or as a three-dimensional path line to show the surgeon a way.

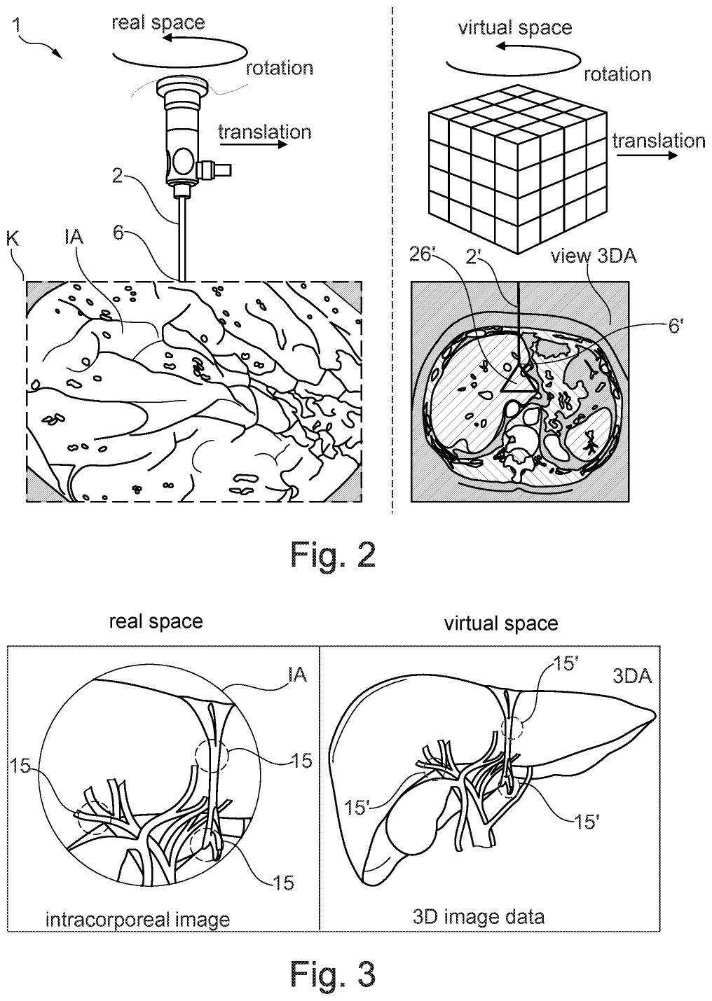

FIG. 2 shows a schematic partial view of an assistance system 1 of a further preferred embodiment with a comparison of the real space and the virtual space to explain the mode of operation. The assistance system 1 differs from the assistance system 1 of FIG. 1 mainly by the selected view of the 3D image data 3DA. The left partial area of FIG. 2 shows the real space and the right partial area shows the virtual space. The real endoscope 2 detects the intracorporeal image IA on the front side via its recording head 6, which serves the surgeon for local navigation. The corresponding virtual endoscope 2′ with its virtual recording head 6′ is displayed in a segment of a CT image (as 3D image data 3DA) at its current position with a superimposed virtual field of view 26′. Thus, for the correlation display K, a segment of the CT image corresponding to the position of the recording head 6 in the superior-anterior direction is used for the view of the 3D image data 3DA. In the view of the 3D image data, the recording head 6′ and its orientation are then displayed schematically in the correct pose. In this way, the surgeon can see at a glance in a simple and intuitive way in the view of the 3D image data 3DA where he/she is globally located in the body of the patient P.

If the endoscope 2 is now moved in FIG. 2 after registration (as already explained for FIG. 1) (see movements arrows for real space), this detected movement is transferred to the virtual endoscope 2′ with the virtual recording head 6′ (see movement arrows for virtual space). On the one hand, this changes the displayed pose of the virtual recording head 6′ and, on the other hand, if the recording head 6 moves in the superior-anterior direction, a segment of the CT image matching this position is displayed. Also, if, for example, the endoscope 2 is rotated about its longitudinal axis, this rotation is transferred to the view of the 3D image data 3DA. For example, in the simplest example, if the endoscope 2 is oriented exactly in the superior-anterior direction and the view of the 3D image data 3DA is also oriented in that direction, the rotation is transferred 1:1 to the view. For example, if the endoscope 2 is rotated 90° clockwise, the view of the 3D image data 3DA is also rotated 90° clockwise. Thus, through the assistance system 1, the intracorporeal image IA is correlated with the (selectable and changeable) view of the 3D image data 3DA and a movement of the endoscope 2 is transferred to the view of the 3D image data 3DA and the view is adjusted accordingly.

In the correlation display, for example, two displays of the intracorporeal image can be included. The first display can be a (split screen) live display in fluorescence mode while the second display shows the ‘normal’ endoscope image. Thus, multiple systems and modes and thus multiple live displays of an intracorporeal image are possible. Likewise, multiple displays of different perspectives of the same 3D image data can be shown in the correlation display.

FIG. 3 schematically shows a preferred embodiment of the assistance system 1 with registration of the intracorporeal image IA to the 3D image data 3DA via three points. In the simplest form, the surgeon selects three characteristic landmarks 15 in the intracorporeal image IA. These three landmarks 15 are reflected as corresponding virtual landmarks 15′ in the 3D image data 3DA. Either the control unit 12 can be adapted to determine the landmarks 15′ automatically or the surgeon can manually determine the three virtual landmarks 15′ in the 3D image data. In this way, the intracorporeal image IA is registered to the 3D image data 3DA as well as the endoscope 2 to the patient P.

FIG. 4 shows a detailed perspective partial view of a rigid endoscope 2 (with rigid shaft) according to an embodiment, which can be used in the assistance device 1 of FIGS. 1 to 3. The endoscope 2 has the handle 4 and the distal recording head 6, which optically detects the intracorporeal image IA (shown here only schematically) in an image direction and provides it digitally to the control unit 12. In the handle 4, the endoscope-internal movement sensor is provided in the form of an inertial measurement unit 18 for forming an inertial navigation system, which detects the movement of the endoscope 2. Using defined geometric parameters between the position of the internal sensor 18 and the tip of the endoscope 2 with the recording head 6, a movement of the recording head 6 can also be calculated by the control unit 12. This movement is then transferred to the 3D image data 3DA in the virtual space accordingly. Also shown in FIG. 4 are three local coordinate systems. A first local coordinate system is that of the handpiece, a second local coordinate system is that of the recording head, and a third local coordinate system is the picture coordinate system of the intracorporeal image. All three coordinate systems can be linked to each other via a suitable (serial) transformation, so that (in particular with corresponding (geometric) data on the endoscope) it is possible to draw conclusions from one coordinate system about another coordinate system.

FIG. 5 shows exemplary different views of the 3D image data, which can be selected by a surgeon via a display selection unit of the assistance system 1 of FIG. 1. This display of the view of the 3D image data 3DA is then rendered in the correlation display K to provide the surgeon with flexible customization and improved orientation. For example, the surgeon can analyze whether there is an offset between the longitudinal axis of the rigid endoscope 2 and the target surgical field if he/she can select a side view, an anterior view, and a top view whose view directions are perpendicular to each other, respectively.

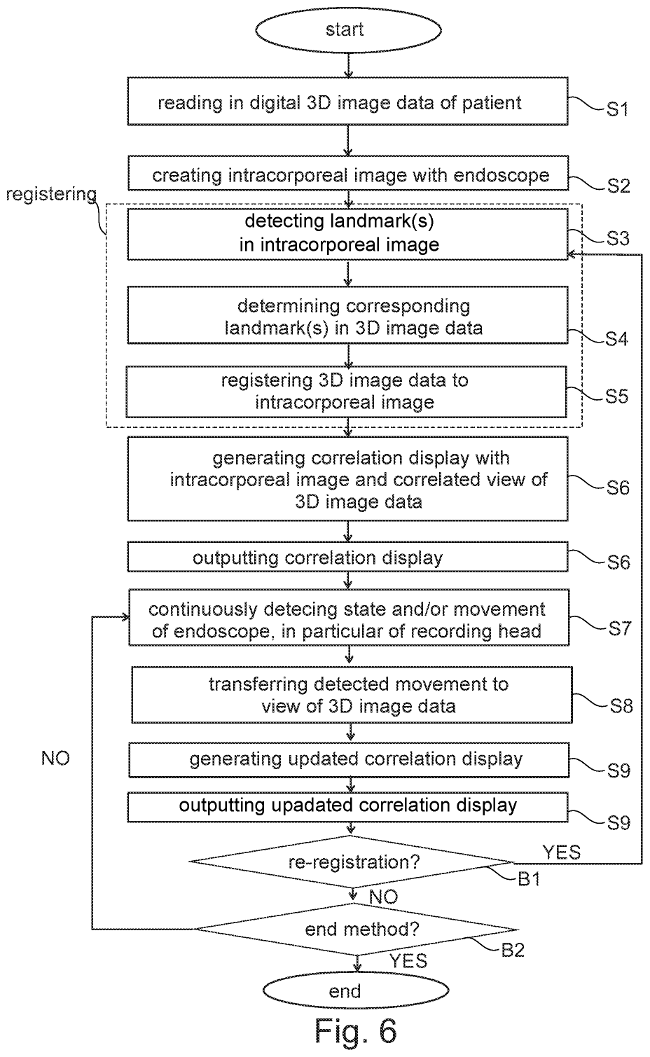

FIG. 6 shows a flowchart of an image display method for central correlated display of two different images, in particular in a surgical assistance system 1 according to a preferred embodiment of the present disclosure. It is noted here that the sequence of steps is also changeable.

In a first step S1, digital 3D image data 3DA, in particular preoperative 3D image data, of a patient P are read in. For example, the 3D image data read in, such as a CT image of the entire body of the patient P, can be stored in a storage unit with corresponding segmentation.

In a next step S2, an intracorporeal image IA is created or detected by the endoscope 2, here by the recording head. It should be noted that the order of steps S1 and S2 is irrelevant, so that the intracorporeal image IA can be taken first and then the reading in, or both steps S1 and S2 can be taken simultaneously.

In a subsequent step S3, landmarks 15 are targeted and detected in the intracorporeal image IA, in particular three landmarks 15. After detecting the landmarks 15, corresponding landmarks 15′ are determined in the 3D image data 3DA in step S4. Via the detected landmarks 15, 15′, the 3D image data 3DA are registered to the intracorporeal image IA in a step S5. The steps S3 to S5 together form the registration step.

In a step S6, a correlation display K with a display of the intracorporeal image IA and a correlated display of a view of the 3D image data 3DA is generated and output, for example by the display device in the form of the OR monitor 8. It can also be said that a transformation correlation of the endoscopic intracorporeal image (endoscopic picture) with the preoperative 3D image data (3D picture) is performed.

In a step S7, a pose and/or a movement of the endoscope 2, in particular via the handling portion 4 indirectly also of the recording head 6, is detected (continuously). This detected movement can be provided as endoscope movement data.

In a step S8, the detected movement of the endoscope 2 with the recording head 6, is transferred to a virtual position and orientation of a virtual recording head 6′ of a virtual endoscope 2′, in the view of the 3D image data 3DA.

In a subsequent step S9, an updated correlation display K is generated with a juxtapositioning of the intracorporeal image IA and the updated view of the 3D image data 3DA, which is displayed by, e.g., the OR monitor 8. In this way, a current, correlated display of the intracorporeal image IA and the view of the 3D image data 3DA is provided to the surgeon.

In a first condition B1 it is checked whether a new registration is to be carried out. If a new registration is to be carried out, the display procedure jumps to step S3 of the detection of landmarks 15.

If no new registration is to be carried out, the method jumps to a second condition B2 and checks whether the procedure is to be terminated. If the method is not to be terminated, the method jumps back to step S7 of the movement detection and a new iteration is executed.

If condition B2 indicates that the method should be terminated, the loop is interrupted and the image display method final is terminated.

For a new registration, the landmarks do not necessarily have to be redefined. In particular, the previously defined landmarks can be used, which are only ‘remeasured’ or correlated.

In particular, the foregoing image display method is stored in the form of instructions on a computer-readable storage medium and, when read and executed by a computer, causes the computer to perform the method steps.

1. A surgical assistance system for use in a surgical intervention on a patient, the surgical assistance system comprising:
a display device for displaying a visual content;
an endoscope comprising an imaging recording head adapted to create an intracorporeal image of the patient;
a data-providing unit adapted to provide 3D image data of the patient that is digital;
a control unit adapted to process the intracorporeal image and the 3D image data;
a registration unit adapted to detect, in the intracorporeal image, an anatomical landmark and/or an anatomical orientation and to determine a corresponding anatomical landmark and/or a corresponding anatomical orientation in the 3D image data, and to register the intracorporeal image with the 3D image data, at least initially, and to register the endoscope relative to the patient; and
a tracking system adapted to continuously detect a position, an orientation and a movement of the endoscope, and to provide the control unit with endoscope movement data,
the control unit being further adapted to generate a correlation display with at least a display of the intracorporeal image and at least a display of a view of the 3D image data, in which the intracorporeal image and the view of the 3D image data are correlated with respect to the endoscope by transferring the endoscope movement data to at least one virtual position and/or orientation of a virtual recording head in the view of the 3D image data for a correlated movement, and the control unit is adapted to visually output the correlation display on the display device.

2. The surgical assistance system according to claim 1, wherein the tracking system is configured as an endoscope-internal tracking system and the endoscope has an endoscope-internal movement sensor for detecting the movement of the endoscope.

3. The surgical assistance system according to claim 1, wherein for the tracking system, the control unit is adapted to determine the movement of the endoscope via an image analysis of moving anatomical structures in the intracorporeal image.

4. The surgical assistance system according to claim 2,
wherein for the tracking system, the control unit is adapted to determine the movement of the endoscope via an image analysis of moving anatomical structures in the intracorporeal image as a first movement determination,
wherein the endoscope-internal movement sensor comprises an acceleration sensor and/or a rotation rate sensor adapted to determine the movement of the endoscope as a second movement determination, and
wherein the control unit is adapted to calculate a final movement determination based on an adjusted mean value or a weighted value calculated from the first movement determination and the second movement determination.

5. The surgical assistance system according to claim 1, wherein the control unit is adapted to recognize at least one medical instrument in the intracorporeal image in a correct position and to display the at least one medical instrument in the view of the 3D image data in the correct position, so that the at least one medical instrument is displayed in the intracorporeal image and in the view of the 3D image data.

6. The surgical assistance system according to claim 1, wherein the registration unit performs a re-registration beyond an initial registration at at least one further point in time in order to further increase an accuracy of a correlation.

7. The surgical assistance system according to claim 1, wherein the endoscope is robot-guided via a robot arm and the tracking system determines the position and/or the orientation and/or the movement of the endoscope via an associated position and/or orientation and/or movement of the robot arm and thus via the robot.

8. The surgical assistance system according to claim 1, wherein the control unit is adapted to display pre-planned medical instruments and/or implants stored in the storage unit in a correct position in the correlation display.

9. The surgical assistance system according to claim 1, wherein the control unit is adapted to display a planned path and/or annotations in the correlation display in order to guide a surgeon to a surgical field.

10. The surgical assistance system according to claim 1, wherein the control unit is adapted to generate the view of the 3D image data, as:
a 3D scene; or
two-dimensional cross-sections relative to a picture-coordinate system of the endoscope and/or along an image axis of the endoscope; or
a virtual endoscope image.

11. An image display method for a central, correlated display of two different images, the image display method comprising the steps of:
reading in 3D image data of a patient;
creating or detecting an intracorporeal image through an endoscope;
detecting at least one landmark and/or anatomical orientation in the intracorporeal image;
determining at least one corresponding landmark and/or at least one corresponding orientation in the 3D image data;
registering the 3D image data to the intracorporeal image via the at least one corresponding landmark and/or the at least one corresponding orientation;
generating a correlation display with at least one display of the intracorporeal image and at least one display of a view of the 3D image data;
continuously detecting a position and/or an orientation and/or a movement of the endoscope;
transferring a detected movement of the endoscope to at least one virtual position and/or at least one virtual orientation of a virtual recording head in the view of the 3D image data; and
generating at least one updated correlation display with the intracorporeal image and an updated view of the 3D image data and outputting the at least one updated correlation display to the display device.

12. The image display method according to claim 11, further comprising the steps of:
determining a deformation of a real anatomical structure based on the at least one landmark and the at least one corresponding landmark, and
transferring the deformation to a virtual anatomical structure in the 3D image data in order to adjust and correct the 3D image data.

13. A computer-readable storage medium comprising instructions which, when executed by a computer, cause the computer to perform the steps of claim 11.

14. A medical sterile space comprising the surgical assistance system according to claim 1.

15. The surgical assistance system according to claim 2, wherein a handling portion and/or the imaging recording head of the endoscope comprises the endoscope-internal movement sensor.

16. The surgical assistance system according to claim 2, wherein the endoscope-internal movement sensor comprises an inertial measurement unit as the endoscope.

17. The surgical assistance system according to claim 5, wherein the control unit is adapted to recognize the at least one medical instrument based on a database stored in the storage unit with geometrically predefined structures of medical instruments.
